(19) United States
(12) Patent Application Publication
(10) Pub. No.: US 2010/0217377 A1
(43) Pub. Date: Aug. 26, 2010

(54) CALCIUM PHOSPHATE COATED STENTS COMPRISING COBALT CHROMIUM ALLOY

(75) Inventor: Manus Tsui, Richmond (CA)

Correspondence Address: RISSMAN HENDRICKS & OLIVERIO, LLP, 100 Cambridge Street, Suite 2101, BOSTON, MA, 02114, US

(73) Assignee: MIV THERAPEUTICS, INC., Vancouver, British Columbia (CA)

(21) Appl. No.: 12/682,422

(22) PCT Filed: Oct. 10, 2008
(86) PCT No.: PCT/CA2008/001795

§ 371 Date: Apr. 9, 2010

Related U.S. Application Data
(60) Provisional application No. 60/978,988, filed on Oct. 10, 2007.

(51) Int. Cl. A61F 2/06; B05D 3/10

(52) U.S. Cl. 623/1.15; 427/2.31

(57) ABSTRACT

Disclosed herein are medical devices, such as stents, coated with calcium phosphate and processes for making the same. The stent can comprise a cobalt chromium alloy that has been treated to improve surface adhesion to the calcium phosphate and/or improve surface finish properties. A pharmaceutically active agent can be present in the calcium phosphate coating.

### RELATED APPLICATION

This application claims the benefit of priority under 35 U.S.C. §119(e) of U.S. Provisional Application No. 60/978,988, filed Oct. 10, 2007, the disclosure of which is incorporated herein by reference.

### FIELD OF THE INVENTION

Disclosed herein are medical devices, such as stents, coated with at least one calcium phosphate, and processes for making the same.

### BACKGROUND OF THE INVENTION

Implantable medical devices are used in a wide range of applications including bone and dental replacements and materials, vascular grafts, shunts and stents, and implants designed solely for prolonged release of drugs. The devices may be made of metals, alloys, polymers or ceramics.

Arterial stents have been used for many years to prevent restenosis after balloon angioplasty (expanding) of arteries narrowed by atherosclerosis or other conditions. Restenosis involves inflammation and the migration and proliferation of smooth muscle cells of the arterial media (the middle layer of the vessel wall) into the intima (the inner layer of the vessel wall) and lumen of the newly expanded vessel. This migration and proliferation is called neointima formation. Stents reduce but do not eliminate restenosis.

Drug eluting stents have been developed to elute anti-proliferative drugs from a non-degradable aromatic polymer coating and are currently used to further reduce the incidence of restenosis. Examples of such stents are the Cypher® stent, which elutes sirolimus, and the Taxus® stent, which elutes paclitaxel. Recently it has been found that both of these stents, though effective at preventing restenosis, cause thromboses (clots) months or years after implantation. These blood clots can be fatal. Late stent thrombosis is thought to be due to the persistence of the relatively toxic drug or the aromatic polymer coating or both on the stent for long time periods. Examination of some of these stents removed from patients frequently shows little or no covering of the stent by the vascular endothelial cells of the vessel intima. This is consistent with the possible toxicity of the retained drugs or non-degradable polymer. The lack of endothelialization may contribute to clot formation.

Accordingly, there remains a need to provide a drug eluting stent having a surface that promotes endothelialization.

### SUMMARY

One embodiment provides a stent comprising a cobalt-chromium alloy and at least one coating covering at least a portion of the stent, wherein the at least one coating comprises at least one calcium phosphate.

Another embodiment provides a method of coating a metal stent, comprising:

acid-etching the metal stent comprising a cobalt-chromium alloy; and

electrochemically depositing at least one calcium phosphate.

### BRIEF DESCRIPTION OF THE DRAWINGS

FIGS. 1A and 1B are photographs at 200× magnification showing two different views of an L605 cobalt chromium stent after the electropolishing step of Example 1;

FIGS. 2A and 2B are photographs at 100× magnification showing two different views of the L605 cobalt chromium stent of Example 1 after coating with hydroxyapatite and crimping;

FIGS. 3A and 3B are photographs at 100× magnification showing two different views of the L605 cobalt chromium stent of Example 1 after expansion;

FIGS. 4A and 4B are photographs at 200× magnification showing two different views of an L605 cobalt chromium stent after the acid-etching step of Example 2;

FIGS. 5A and 5B are photographs at 200× magnification showing two different views of the L605 cobalt chromium stent of Example 2 after coating with hydroxyapatite and crimping; and

FIGS. 6A and 6B are photographs at 200× magnification showing two different views of the L605 cobalt chromium stent of Example 2 after expansion.

### DETAILED DESCRIPTION

One embodiment provides a stent comprising a cobalt-chromium alloy and at least one coating covering at least a portion of the stent, wherein the at least one coating comprises at least one calcium phosphate.

Cobalt-chromium alloys are being recognized as a viable material for stents, offering a more biocompatible material compared to stainless steel. Stents comprising cobalt-chromium alloys can have a higher radial strength and a higher radiopacity than stainless steel. A high elastic modulus and density allows stents comprising cobalt-chromium alloys to have thinner struts and a lower profile that is useful for small diameter lumens.

However, the presence of secondary phase metallic precipitates in this alloy can reduce the adhesion of coatings to the metal surface and can affect the mechanical properties of the stent, including one or more of grain coarsening that affects the surface finish, yield strength (which can influence crimping recoil and balloon expansion pressure), fatigue resistance, and expansion uniformity. Moreover, the precipitates themselves present the potential of being released into the blood stream. Such precipitates can be metal carbides or intermetallic compounds such as CoW intermetallic compounds. For example, precipitates on an L605 stent can include carbides such as at least one of M₇C₃, M₂₃C₆, M₆C, where M can be Cr and/or W, most likely W. Intermetallic compounds can include CO₃W (α and β phases) and Co₇W₆.

Accordingly, in one embodiment, the cobalt-chromium surface of the stent is pretreated with an acid etch to reduce or even eliminate the presence of precipitates and ultimately improve one or more of the stent properties listed above.

In one embodiment, a cobalt-chromium stent is acid etched by immersion of the stent in an acid solution before depositing the calcium phosphate coating. In one embodiment, an acid solution has a pH of less than 7, such as a pH of less than 6.5, less than 5, less than 4, less than 3, or even less than 2. In one embodiment, the acid solution has an acid concentration of at least 25%, such as an acid concentration of at least 50%, or an acid concentration of at least 90%. In one embodiment, the acid etch solution comprises an aqueous solution of hydrochloric acid at a concentration of from about 0.5% to about 39% and sulfuric acid at a concentration of about 0.5% to about 97%. In another embodiment, the acid solution contains 4.5% to 18% hydrochloric acid and 12.25% to 50% sulfuric acid. In yet another embodiment, the acid solution comprises a mixture of hydrochloric acid and sulfuric acid in a ratio ranging from 3:1 to 1:10, from 3:1 to 1:3, from 2:1 to 1:3, even from 2:1 to 1:2, such as 1:1 mixture of hydrochloric acid and sulfuric acid.

The stent can be immersed in the acid solution for a period of time ranging from 1 second to 1 week, such as a period of time ranging from 15 minutes to 24 hours, or from 15 minutes to 2-3 hours. In another embodiment, acid etch temperatures can range from 0° C. to 100° C., such as a temperature ranging from 25° C. to 80° C., or at room temperature.

In one embodiment, the surface of the acid-etched stent is free or substantially free of secondary phase metallic precipitates, such as tungsten-containing precipitates (e.g., tungsten carbides and intermetallic compounds) disclosed herein. In another embodiment, the surface of the acid-etched stent has less than 50%, or even less than 25%, the amount secondary phase metallic precipitates than the surface of a stent comprising cobalt chromium alloy that has not been pretreated as described herein.

Calcium phosphates may be used to coat devices made of metals or polymers to provide a more biocompatible surface. Calcium phosphates are often desirable because they occur naturally in the body, are non-toxic and non-inflammatory, and are bioabsorbable. Such devices or coatings may serve as a matrix for cellular and bone in-growth in orthopedic devices or to control the release of a therapeutic agent from any device. In the field of vascular stents, calcium phosphate coatings can be attractive because they can provide a biocompatible surface that can be rapidly covered by the endothelial cells of the vascular intima. In contrast, polymer coatings of prior art drug eluting stents do not promote endothelialization. Alternatively, the calcium phosphate can be of a bioresorbable form, resulting in a bare metal stent that avoids the problems of late thrombosis found with commercially available polymer-coated stents.

In one embodiment, the coated stent is a drug eluting stent in which at least one pharmaceutically active agent impregnates the porous calcium phosphate, e.g., the agent is deposited on the calcium phosphate and/or in the pores of the porous calcium phosphate. In one embodiment, the coating has a thickness of no more than 2 μm, such as a thickness of no more than 1 μm or no more than 0.5 μm. In one embodiment, the calcium phosphate in the coating is porous and has a porosity volume ranging from 30 to 70% and an average pore diameter ranging from 0.3 μm to 0.6 μm. In other embodiments, the porosity volume ranges from 30 to 60%, from 40 to 60%, from 30 to 50%, or from 40 to 50%, or even a porosity volume of 50%. In yet another embodiment, the average pore diameter ranges from 0.4 to 0.6 μm, from 0.3 to 0.5 μm, from 0.4 to 0.5 μm, or the average pore diameter can be 0.5 μm. Calcium phosphates displaying various combinations of the disclosed thicknesses, porosity volumes or average pore diameters can also be prepared.

These thickness, porosity, and pore diameter ranges can result in a flexible calcium phosphate coating that stays adhered to the stent even during mounting, crimping, and expansion of the stent. A typical mounting process involves crimping the mesh-like stent onto a balloon of a catheter, thereby reducing its diameter by 75%, 65%, or even 50% of its original diameter. When the balloon mounted stent is expanded to place the stent adjacent a wall of a body lumen, e.g., an arterial lumen wall, the stent, in the case of stainless steel, can expand to up to twice or even three times its crimped diameter. For example, a stent having an original diameter of 1.6 mm can be crimped to a reduced diameter of 1.0 mm. The stent can then be expanded from the crimped outer diameter of 1.0 mm to an outer diameter of 3.0, 3.5 or even 4.5 mm.

Under these process conditions, thicker or less porous coatings can be brittle, can develop significant cracks, and/or can shed particles or flakes. In one embodiment, the coating is well bonded to the substrate and does not form significant cracks and/or does not flake off from the stent during mounting on a balloon catheter and placement and expansion in a body lumen. In one embodiment, a coating that does not form significant cracks can have still present minor crack formation so long as it measures less than 300 nm, such as cracks less than 200 nm, or even less than 100 nm.

In another embodiment, the coating can withstand a fatigue test to meet the requirements as per the “FDA Draft Guidance for the Submission of Research and Marketing Applications for Interventional Cardiology Devices” that demonstrates the safety of the device from mechanical fatigue failures for at least one year of implantation life. The test is designed to simulate the stent fatigue due to the expansion and contraction of the vessel in which it is implanted. For example, the coated stents can be tested in phosphate buffer saline (PBS) at 37° C.±3 C, with a EnduraTec fatigue testing machine (ElectroForce® 9100 Series, EnduraTec System Corporation, Minnesota, USA) that can simulate the equivalent of one year of in-vivo implantation, e.g., approximately 40 million cycles of fatigue stress, which simulates heart beat rates from 50-100 beats per minute.

In one embodiment, the porosity volume and pore sizes in calcium phosphate coatings can be selected to act as reservoirs for controlling the release of pharmaceutically active agents. In one embodiment, the pharmaceutically active agent is selected from those agents used for the treatment of restenosis, e.g., anti-inflammatory agents, anti-proliferatives, pro-healing agents, gene therapy agents, extracellular matrix modulators, anti-thrombotic agents/anti-platelet agents, antiangioplastic agents, antisense agents, anticoagulants, antibiotics, bone morphogenetic proteins, integrins (peptides), and disintegrins (peptides and proteins), such as those agents disclosed in U.S. Provisional Application No. 60/952,565, filed Jun. 7, 2007, the disclosure of which is incorporated herein by reference. Other exemplary classes of agents include agents that inhibit restenosis, smooth muscle cell inhibitors, immunosuppressive agents, and anti-antigenic agents. Exemplary drugs include sirolimus, paclitaxel, tacrolimus, heparin, pimecrolimus, midostaurin, imatinib mesylate (gleevec), and bisphosphonates.

The release of drugs from prior art polymer coatings for drug eluting stents depend substantially on the rate of diffusion of the drug through the polymer coating. While diffusion may be a suitable mechanism for drug release, the rate of drug release from the polymer coating may be too slow to deliver the desired amount of drug to the body over a desired time. As a result, a significant amount of the drug may remain in the polymer coating. In contrast, one embodiment disclosed herein allows selecting the porosity volume and average pore size to provide pathways for the drug be released from the coating, thereby increasing the rate of drug release compared to a polymer coating. In another embodiment, these porosity properties can be tailored to control the rate of drug release. In one embodiment, at least 50% of the agent is released from the stent over a period of at least 7 days, or at least 10 days and even up to a period of 1 year. In another embodiment, at least 50% of the agent is released from the stent over a period ranging from 7 days to 6 months, from 7 days to 3 months, from 7 days to 2 months, from 7 days to 1 month, from 10 days to 1 year, from 10 days to 6 months, from 10 days to 2 months, or from 10 days to 1 month.

In one embodiment the calcium phosphate coating may be deposited by electrochemical deposition (ECD) or electrophoretic deposition (EPD). In another embodiment the coating may be deposited by a sol gel (SG) or an aero-sol gel (ASG) process. In another embodiment the coating may be deposited by a biomimetic (BM) process. In another embodiment the coating may be deposited by a calcium phosphate cement process. In one embodiment of a cement process, a calcium phosphate cement coating with about a 16 nm pore size, a porosity of about 45%, and containing a dispersed or dissolved therapeutic agent, is applied to a stent previously coated with a sub-micron thick coating of sol-gel hydroxyapatite as previously described in U.S. Pat. No. 6,730,324, the disclosure of which is incorporated herein by reference. The resulting coating encapsulates the agent, and agent release is controlled by the dissolution of the coating.

The electrochemical deposition can be varied to achieve the desired porosity features. Variables include current density (e.g., ranging from, 0.05-2 mA/cm² such as 0.5-2 mA/cm²), deposition time (e.g., 2 minutes or less, or 1 minute or less), and electrolyte composition, pH, and concentration. Such variables can be manipulated as discussed in Tsui, Manus Pui-Hung, “Calcium Phosphate Coatings on Coronary Stents by Electrochemical Deposition,” M.A.Sc. diss., University of British Columbia, University, 2006, the disclosure of which is incorporated herein by reference.

In one embodiment, the electrochemically deposited calcium phosphate is a mixed-phase coating comprising partially crystalline hydroxyapatite and dicalcium phosphate dihydrate. Substantially pure hydroxyapatite can be achieved by subjecting the coated stent to the second alkaline solution, followed by heating the coated stent at a temperature ranging from 400° C. to 750° C., such as a temperature ranging from 400° C. to 600° C. The phase can be monitored by x-ray diffraction, or other methods known in the art. In one embodiment, the method results in a porous calcium phosphate, such as a porous hydroxyapatite. The porous calcium phosphate (e.g., porous hydroxyapatite) can be stable in body fluid for at least one year, or even for at least two years, thereby allowing sufficient time for endothelialization to occur on the calcium phosphate surface.

In one embodiment a composition ratio of calcium salt and phosphate salt is selected to give a desired calcium phosphate after deposition. For example, a Ca/P ratio can be selected to range from 1.0 to 2.0.

In another embodiment, the release rate of a therapeutic agent by a calcium phosphate coating can be controlled by the bioresorption or biodegradation of the calcium phosphate itself. Bioresorption and biodegradation can be generally controlled by at least one or more of the following factors: (1) physiochemical dissolution, e.g., degradation depending on the local pH and the solubility of the biomaterial; (2) physical disintegration, e.g., degradation due to disintegration into small particles; and, (3) biological factors, e.g., degradation cause by biological responses leading to local pH decrease, such as inflammation.

In one embodiment, the coating comprises at least one calcium phosphate selected from octacalcium phosphate, α- and β-tricalcium phosphates, amorphous calcium phosphate, dicalcium phosphate, calcium deficient hydroxyapatite, and tetracalcium phosphate, e.g., the coating can comprise a pure phase of any of the calcium phosphates or mixtures thereof, or even mixtures of these calcium phosphates with hydroxyapatite. In one embodiment, the at least one calcium phosphate comprises hydroxyapatite.

In one embodiment at least one calcium phosphate is deposited on a stent as a single layer. In another embodiment a single calcium phosphate is deposited as multiple layers. In another embodiment a calcium phosphate is deposited in one layer and one or more layers of one or more other calcium phosphates can be successively deposited over the first layer.

Another embodiment provides a method of treating at least one disease or condition associated with restenosis, using either a stent coated with at least one porous calcium phosphate that is stable to resorption, allowing the drug to be released through the pores of the calcium phosphate. In another embodiment, the stent is coated with a porous calcium phosphate that is resorbed relatively quickly to release the drug that impregnates the calcium phosphate.

### EXAMPLES

### Example 1

### Control

This Example describes deposition of hydroxyapatite on a stent comprising a cobalt chromium alloy without the pretreatment process described herein. The hydroxyapatite deposition is also disclosed in Tsui, Manus Pui-Hung, “Calcium Phosphate Coatings on Coronary Stents by Electrochemical Deposition,” M.A.Sc. diss., University of British Columbia, University, 2006, the disclosure of which is incorporated herein by reference.

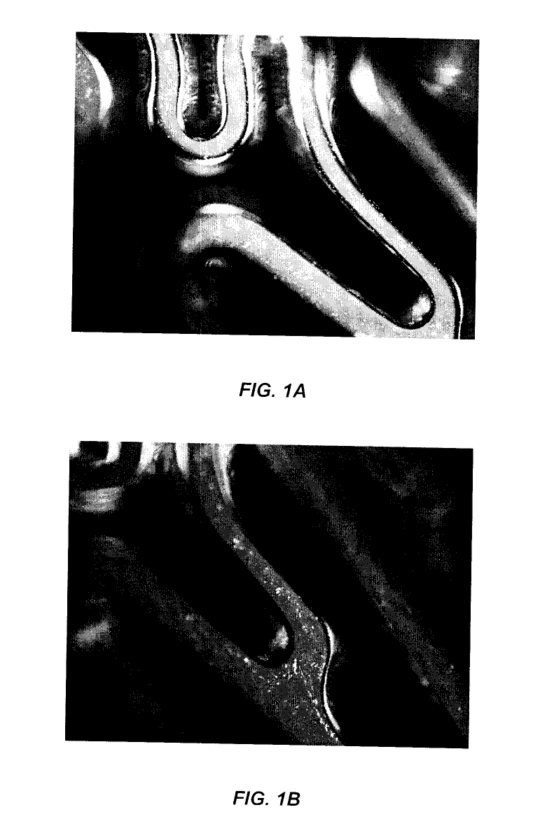

The stent used was a L605 cobalt chromium stent (cobalt-chromium-tungsten-nickel alloy, MIV Therapeutics, Inc.) measuring 19 mm in length and a 1.6 mm outer radius. The stent surface was electro-polished, then cleaned in ultrasonic bath, with distilled water and then with ethyl alcohol. FIGS. 1A and 1B are photographs of two different portions of the stent after the electropolishing method. From these photographs, numerous precipitates are visible on the surface of the stent.

Electrochemical deposition of calcium phosphate was performed with 400 mL of electrolyte consisting of 0.02329M Ca(NO₃)₂.4H₂O and 0.04347M NH₄H₂PO₄ at 50° C. The pretreated stent was used as the cathode and a platinum cylinder was used as the anode. When a 0.90 mA current was applied for 60 seconds, a thin film of hydroxyapatite coating was deposited on the stent. In other embodiments, a current density of 0.05-2 mA/cm², e.g., 0.5-2 mA/cm², can be used depending on the stent size. The coated stent was then washed with running distilled water for 1 minute and air dried for 5 minutes.

The stent was then subjected to a post-treatment process of soaking the stent in 0.1N NaOH (aqueous) solution at 75° C. for 24 hours, followed by an ultrasonic cleaning with distilled water and a heat treatment at 500° C. for 20 minutes. The final coating had a thickness of ˜0.5 μm and uniformly covered the stent.

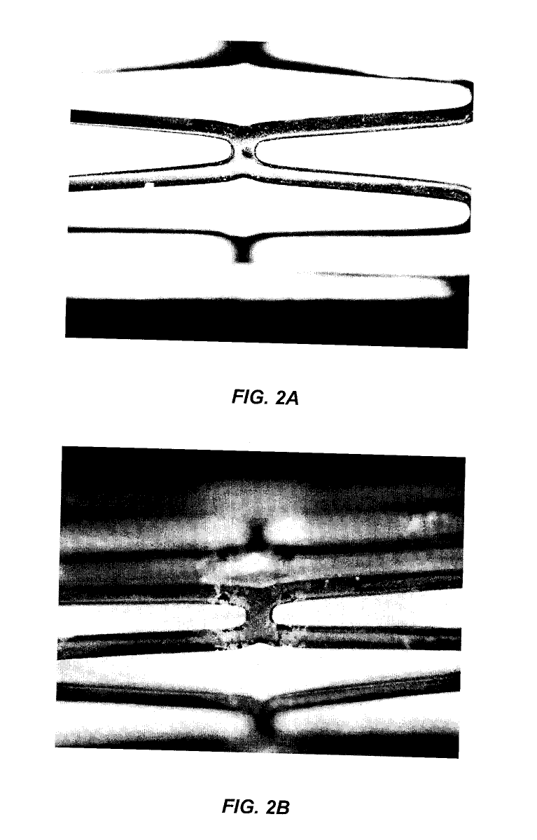

The stent was crimped from an initial outer diameter of 1.6 mm to 1.0 mm with a SC775 Stent Crimping machine from Machine Solution, Inc. FIGS. 2A and 2B are photographs of two different portions of the stent after crimping. It can be seen that the hydroxyapatite coating has flaked and delaminated from portions of the stent due to insufficient adhesion and undesirable surface finish due to the presence of precipitates.

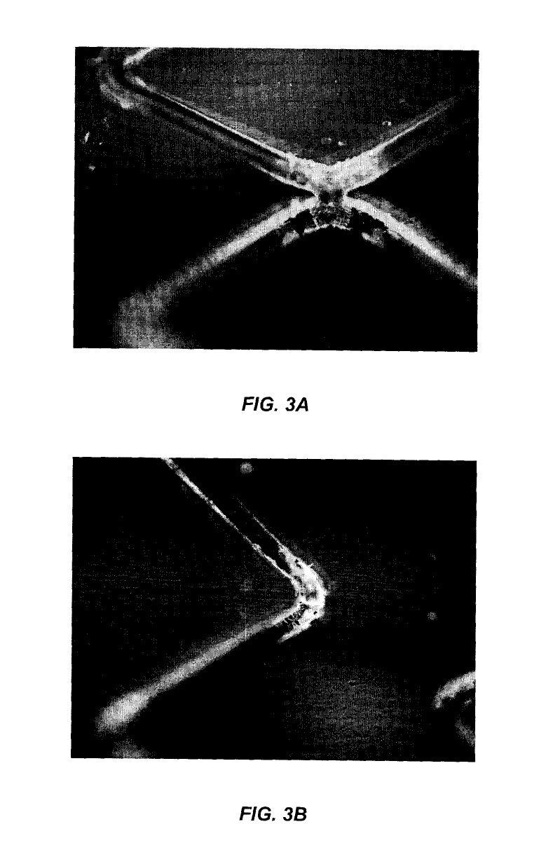

An expansion test was performed after the crimping process. An Encore™ 26 INFLATION DEVICE KIT was used to inflate the catheter to 170 psi, and the stent was expanded from the crimped outer diameter of 1.0 mm to 3.5 mm. FIGS. 3A and 3B are photographs of two different portions of the stent after expansion, showing even greater flaking and delamination than that of FIGS. 2A and 2B.

### Example 2

This Example describes coating a cobalt-chromium alloy stent after an acid-etching pretreatment.

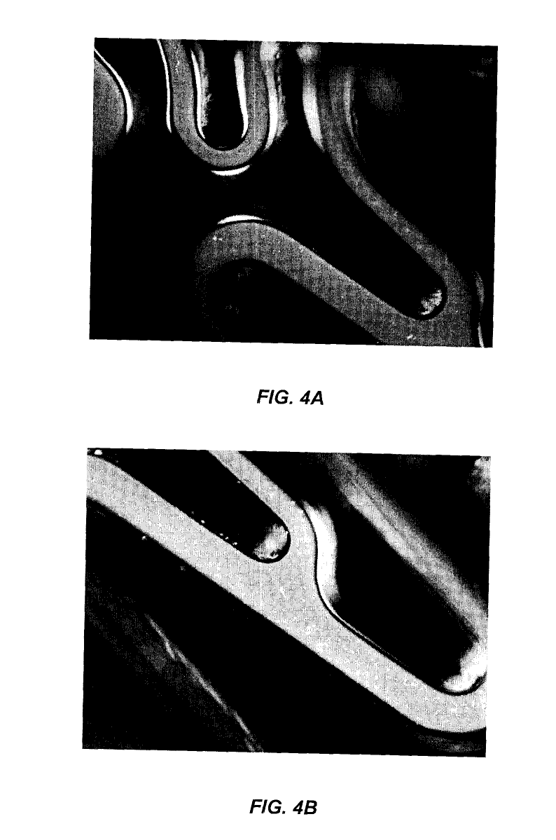

A concentrated acid etch reagent was made by mixing 95-98% sulfuric acid and 36-40% hydrochloric acid in 1:1 proportion. A 25% acid etch working solution was made by diluting the 1:1 reagent with HPLC grade water (all % concentrations are volume/volume). The working solution was 4.5% hydrochloric acid, 12.25% sulfuric acid and 83.25% HPLC grade water. A L605 cobalt-chromium alloy stent was cleaned by sonicating in distilled water and then in ethyl alcohol, followed by rinsing with ethyl alcohol and air drying. The dried stent was immersed in 5 mL of the working solution in a capped pyrex test tube and gently agitated at 25° C. in a rotary water bath for 1 hour. The stent was removed, rinsed exhaustively in HPLC grade water and air dried. FIGS. 4A and 4B are photographs of the surface of the acid-etched stent. It can be seen that the precipitate formation on the surface finish is greatly reduced when comparing to the non-acid-etched stent of Example 1, as shown in FIGS. 1A and 1B.

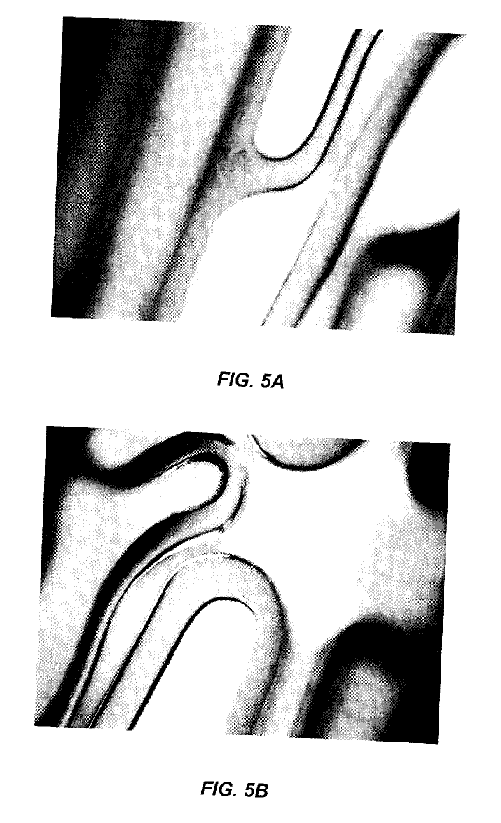

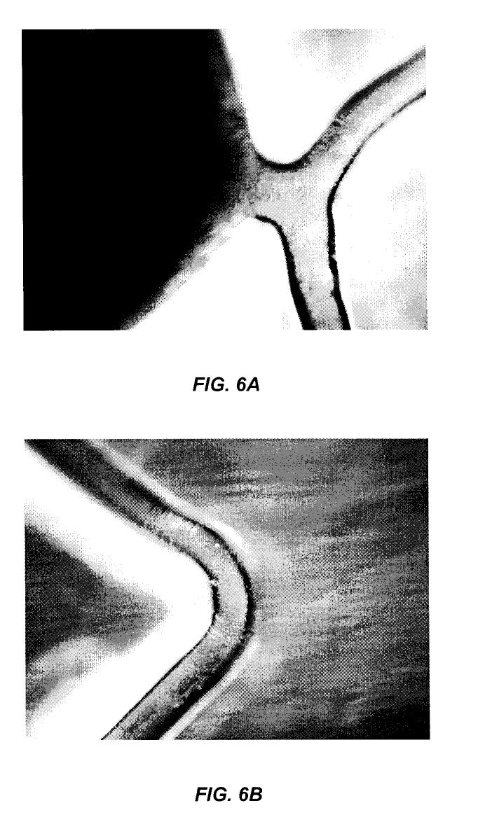

The acid etched cobalt-chromium stent was coated with a calcium phosphate by electrochemical deposition as described in Example 1, followed by the same crimping and expansion processes. FIGS. 5A and 5B are photographs of two different portions of the stent showing the results of the crimping. No delamination can be observed. Similarly, FIGS. 6A and 6B are photographs of two different portions of the stent, showing no observable delamination after stent expansion.

It can be seen that the acid-etching processes result in improved surface finish, which can translate to improved mechanical properties and/or coating adhesion and thus, coating stability and integrity.

1. A stent comprising a cobalt-chromium alloy and at least one coating covering at least a portion of the stent, wherein the at least one coating comprises at least one calcium phosphate.

2. The stent of claim 1, wherein the at least one calcium phosphate is hydroxyapatite.

3. The stent of claim 1, wherein the at least one calcium phosphate is a porous calcium phosphate having a porosity volume ranging from 30-60% and an average pore diameter ranging from 0.3 μm to 0.6 μm.

4. The stent of claim 3, the at least one coating further comprising at least one pharmaceutically active agent impregnating the porous calcium phosphate.

5. The stent of claim 3, wherein the at least one coating is free of a polymeric material.

6. The stent of claim 3, wherein the at least one calcium phosphate coats an acid-etched surface of the stent.

7. A method of coating a metal stent, comprising:
acid-etching the metal stent comprising a cobalt-chromium alloy; and
electrochemically depositing at least one calcium phosphate on the acid-etched stent.

8. The method of claim 7, wherein the acid-etching step comprises subjecting the metal stent to an acid solution.

9. The method of claim 8, wherein the acid solution comprises at least one acid selected from sulfuric acid and hydrochloric acid.

10. The method of claim 8, wherein the acid solution has an acid concentration of at least 25%.

11. The method of claim 8, wherein the acid solution comprises at least 4% hydrochloric acid by volume.

12. The method of claim 8, wherein the acid solution comprises at least 12% sulfuric acid by volume.

13. The method of claim 8, wherein the acid solution comprises a mixture of hydrochloric acid present in an amount ranging from 0.5%-39% by volume and sulfuric acid present in an amount ranging from 0.5%-97% by volume.

14. The method of claim 8, wherein the acid solution comprises a mixture of hydrochloric acid and sulfuric acid in a ratio ranging from 3:1 to 1:10.

15. The method of claim 7, wherein the at least one calcium phosphate is hydroxyapatite.
